(19) United States
(12) Patent Application Publication
(10) Pub. No.: US 2007/0038035 A1
(43) Pub. Date: Feb. 15, 2007

(54) Method and system for screening and indicating individuals with hidden intent

(75) Inventors: Sharon Ehrlich, Beit Herut (IL); Eli Barnea, Haifa (IL); Shlomo Breznitz, Haifa (IL); Boaz Ganor, Herzelia (IL); Zipora Alster, D.N. Hamovil (IL); Ehud Givon, Herzlia (IL)

Correspondence Address: BROWDY AND NEIMARK, P.L.L.C.;624 NINTH STREET, NW, SUITE 300, WASHINGTON, DC, 20001-5303, US

(73) Assignee: W.E.C.U. TECHNOLOGIES LTD., Caesaria (IL)

(21) Appl. No.: 10/573,820

(22) PCT Filed: Sep. 28, 2004
(86) PCT No.: PCT/IL04/00904

§ 371 Date: Mar. 28, 2006

(30) Foreign Application Priority Data
Oct. 1, 2003 (IL) 158207

(51) Int. Cl. A61B 5/00

(52) U.S. Cl. 600/300; 128/920

(57) ABSTRACT

A method and system and for screening and indicating individuals with hidden intent, wherein the system comprises: a) a working place for fulfillment of at least one stated assignment; b) a stimuli exposure block configured to exposure at least one stimuli to said working place within at least one exposure timeframe while said timeframe is contemporary with individual's fulfillment of at least one stated assignment and said stimulus does not require an active direct reaction from the individual and is intended to cause a psycho-physiological reaction; c) sensors and measuring tools block configured to register individual's psycho-physiological characteristics giving rise to registered parameters; and a processor configured to compare the registered parameters or derivatives thereof with a test criterion and to facilitate indication of hidden intent in case of predefined discrepancy.

### FIELD OF THE INVENTION

This invention, in general, deals with automated screening and indicating individuals with hidden, e.g. malicious, intent, in the course of the preparations for acting, while committing the act, and thereafter.

In one embodiment, the invention may be implemented for spot-screening procedures, e.g. of passengers and crowds; progressive screening procedures, e.g. of applicants for employment in sensitive organizations; and continuous screening and identification of suspects, e.g. locating suspect employees within a sensitive organization.

### BACKGROUND OF THE INVENTION

From the beginning of society, it has always been man's desire to have the ability to detect those who are seeking to cause harm, and who might pose a major security threat to their fellowmen.

The present invention relates to the problem of identifying signs of attempted deception or other hidden intent. For example, to reduce risk of terrorism or other malfeasance by enhancing the access control at sensitive heavy-traffic locations (e.g. air and sea ports, border crossings, business and shopping centers) as well as to assure trustworthiness of human resources in sensitive organizations.

Many systems were developed to provide a solution, such as:
i. Variable sensors and sniffers (chemical, metal, electromagnetic and X-ray radiation, biometric, physiological, behavioral, etc.) are limited by dependence upon intelligence or other early information and do not reveal persons with malicious intent that do not carry suspicious items, or do carry hazardous materials and camouflaged objects that the inspection is unable to uncover.
ii. Human interpreted questioning of individuals, for example, by airline security personnel, and even more sophisticated interrogation and “profiling” has a low probability of uncovering trained persons.
iii. Polygraphic (“lie-detection”) technologies where the polygraph senses the changes in physiological signals to indicate a person's level of anxiety as he or she answers questions. A trained polygraphist can use these measures of anxiety to identify suspicious answers. However, certain specialists believe that of the polygraph has few shortcomings, for instance the accuracy is not a sufficient in “screening mode” for testing non-specific incidents and individuals. (See, e.g. “The polygraph and lie detection”, 2003, pp. 1-9, National Academy of Science, USA).
iv. Polygraph-like technique for psycho-physiological detection of deception. The goal of all of these techniques is to detect deception by analyzing signals of changes in the body that cannot normally be detected by human observation.

The known improvements of the classical polygraph are directed to:
extending measuring of psycho-physiological parameters (e.g. U.S. Pat. No. 4,941,477 (Farwell) U.S. Pat. No. 5,137,027 (Rosenfeld) and later U.S. Pat. No. 6,754,524 (Johnson) uses electro-encephalography to measure P3 brain-waves to perform lie detection; U.S. Pat. No. 5,507,291 (Stirbl et. al.) shows remote measuring parameters such as blood pressure, pulse rate, pupil size, respiration rate and perspiration level by transmitting a generated waveform at a remotely located subject and analyzing the result; U.S. Pat. No. 5,771,261 (Anbar) describes tele-thermometric psychological evaluation by monitoring changes in skin perfusion induced by the autonomic nervous system; U.S. Pat. No. 5,774,571 (Marshall) uses a pen incorporating a trembling sensor to ascertain likely signs of stress and therefore deception on the part of the person writing with the pen; U.S. Pat. No. 5,853,005 (Scanlon) describes a hydrophone fitted into a seat to measure voice stress levels, heart and breath rate, and body temperature for surreptitious interrogation or identification);
advanced analysis of the measured results (e.g. U.S. Pat. No. 5,876,334 (Levi) and later U.S. Pat. No. 6,757,559 (Cohen) teach lie detection based on analyzing response time to specific, carefully formed questions) and
the development of new stimuli designed to evoke the psycho-physiological response (e.g. U.S. Pat. Appln. No. 2004/0143170 (DuRousseau) describing a virtual reality system presenting cognitive stimuli and registering the evoked response).

### SUMMARY OF THE INVENTION

There is a need in the art to provide a system and method which substantially reduces or eliminates the drawbacks of hitherto known solutions.

The invention includes the technical teaching of an effective method and system for reliable screening and indicating individuals with malicious and other hidden intent. In contrast to polygraph-like methods, the stimuli are exposed in parallel with some special or routine assignment executed by a tested individual and do not require any active reaction by the individual.

The invention includes teaching of embodiments of the “dual task” procedure while the individual is focused on the primary task (assignment) and the secondary task (stimuli) is out of the individual's focus. The present method and system enable to “stick out” individuals with hidden intent and reveal them to the screening system, without them being aware of it doing so, or being able to control and avoid it.

In the certain embodiments the invention may be implemented for different screening scenarios such as, for example:

i. spot screening, operating on-the-spot measures with one-time subject-system encounter; this type of screening is extremely important, e.g. for access control of passengers and/or crowds;

ii. progressive screening, operating progressive repeated measures including several tests, performed during a prolonged testing event of an individual; this may be useful, e.g., for screening of applicants for employment in sensitive organizations;

iii. continuous screening operating continuous repeated measures while creating a base-line. This routine, sustained-testing process allows a thorough investigation that is practically unperceivable by the subject, e.g., applicable for security screening and pointing out deceptive employees in sensitive organizations.

The invention is not bounded to these screening schemes.

According to one aspect of a certain embodiment of the present invention there is provided a method for screening and indicating individuals with hidden, e.g. malicious intent; the method further including the steps of:
a. exposing an individual to at least one stimulus within at least one exposure timeframe while said timeframe is contemporary with individual's fulfillment of at least one stated assignment and said stimulus does not require an active direct reaction of the individual and is intended to cause a psycho-physiological reaction;
b. registration of individual's psycho-physiological characteristics giving rise to registered parameters;
c. comparing the registered parameters or derivatives thereof with a test criterion;
d. providing an indication of hidden intent in case of predefined discrepancy.

The stimuli may be targeted to specific malicious group(s) or malicious issue(s) and create response that can be measured. In contrast to polygraph-like methods, such approach may be based also on predefined generic details and characteristics of malicious groups/issues and not necessarily on specific event information. The stimuli may not form a part of the assignment.

According to further features in the described certain embodiment said stimuli may be any one of or any combination of open or concealed, stressing or relaxing, creating conscious or sub-conscious reaction, relevant and irrelevant, targeted or general stimuli selected and provided with or without human intervention, provided via physical and/or virtual media. Said stimuli do not require an active direct reaction of the tested individual (e.g. there is no need of an active action as to answer a question or to select a specific picture in reaction to stimuli), but do prompt a measurable psycho-physiological (incl. biometric and behavioral) reaction.

According to further features in the described certain embodiment, stimuli within said exposure timeframe may be grouped in dedicated sets, e.g. neutral, control, manipulating, authentication, relaxing set, etc.

Said exposure timeframes may start simultaneously with individual's fulfillment of assignment(s) or later and finish simultaneously or before end of the assignment. The time of appearance and duration of exposure time frames as well as type and time of appearance of the stimuli within said exposure time frame(s) may be random (pseudo random) or scheduled.

Said fulfillment of stated assignment may include one or several assignments, continuous or with timebreak(s), identical or of different content and/or nature.

According to further features in the described certain embodiment, said test criterion may include a test pattern. The test pattern may be predefined, based on a prior knowledge of characteristics of a threat group and/or neutral group as well as based on prior registered characteristics of the tested individual. The test pattern may also be adapted to the tested individual according to the registered parameters. By way of example, said adaptive pattern may be a pattern comprising a predefined set of parameters to be measured, e.g. Galvanic Skin Response (GSR) before and after exposure, whereas the values of these predefined parameters are registered during the test. The pattern may be based on the measured psycho-physiological response to exposure as well as on the measured parameters characterizing fulfillment of the assigned task. The pattern may be further used as a baseline for correlation analysis with psycho-physiological (biometric and behavioral) parameters registered. during some selected period of time.

According to another aspect of the certain embodiment of the present invention there is provided a method for screening and indicating individuals with hidden malicious and other intent comprising:
a. exposing the individuals to at least two sets of stimuli within at least one exposure timeframe, while the first set of stimuli are dedicated to cause in an individual a response known in advance, the second set is dedicated to indicate a hidden intent;
b. registration of characteristics of response to such exposure, giving rise to registered parameters;
c. comparing the parameters or derivatives thereof registered during the exposure of the first set with the known in advance individual's response;
d. comparing the parameters or derivatives thereof registered during the exposure of the second set with a test criterion;
e. providing an indication of hidden intent in case of predefined discrepancy.

According to further features in the described certain embodiment said first and second set of stimuli may be any one or any combination of open or concealed, stressing or relaxing, creating conscious or sub-conscious reaction, relevant and irrelevant, targeted or general stimuli selected and provided with or without human intervention, provided via physical and/or virtual media and prompting any measurable psycho-physiological reaction.

According to further features in the described certain embodiment said exposure timeframe(s) may be contemporary with individual's fulfillment of special or routine assignment(s). Said exposure timeframe may start simultaneously with individual's fulfillment of assignment(s) or later and finish simultaneously or before end of the assignment. The first and second set of stimuli can be exposed in different timeframes. The time of appearance of the timeframes as well as of said sets of stimuli within the exposure time frame(s) may be random (pseudo random) or scheduled.

According to further features in the described certain embodiment said known in advance response for the first set of stimuli may be based on prior registered parameters of the tested individual.

According to further features in the described certain embodiment said test pattern may be adaptive wherein the types of parameters are predefined and their values (or part of them) are drawn from the registered parameters of the individual's response.

According to another aspect of the certain embodiment of the present invention there is provided a system for screening and indicating on individuals with hidden intent comprising:

a. a working place for fulfillment of at least one stated assignment;
b. a stimuli exposure block for exposure of at least one stimuli to said working place;
c. a sensors and measuring tools block for registration of individual's psycho-physiological characteristics giving rise to registered parameters;
d. a databases block for storing data related to screening and indication setup and execution whereas said databases block includes:
   i. a tools and sensors database comprising data about at least one stimuli exposure tool and at least one sensors and measuring tools;
   ii. a stimuli database comprising at list one stimulus;
   iii. an exposure procedures database comprising at least one exposure scenario and accordingly psycho-physiological characteristics to be registered;
   iv. an evaluation procedures database comprising at least one test criterion and at least one predefined discrepancy for said exposure scenario as well as at least one rule for evaluation of discrepancy between said registered parameters and test criterion.
e. a processing block for processing and management of data stored in said database block.

### BRIEF DESCRIPTION OF THE DRAWINGS

In order to understand the invention and to see how it may be carried out in practice, a certain embodiment will now be described, by way of non-limiting examples only, with reference to the accompanying drawings, in which:

FIG. 1 illustrates a generalized block diagram of exemplary system architecture, in accordance with an embodiment of the invention.

FIG. 2 illustrates a flow diagram showing the principal steps for operating the setup for the test in accordance with an embodiment of the invention;

FIG. 3 illustrates a flow diagram showing the principal steps of operating the test in accordance with an embodiment of the invention;

FIG. 4 illustrates a flow chart of generalized time sequence of test operating, in accordance with an embodiment of the invention;

FIG. 5 illustrates another flow chart of generalized time sequence of test operating, in accordance with another embodiment of the invention;

FIG. 6 illustrates another flow chart of generalized time sequence of test operating in accordance with another embodiment of the invention;

FIG. 7 illustrates a flow diagram showing the principal steps of operating the test in a described experience.

### DESCRIPTION OF DETAILED EMBODIMENT

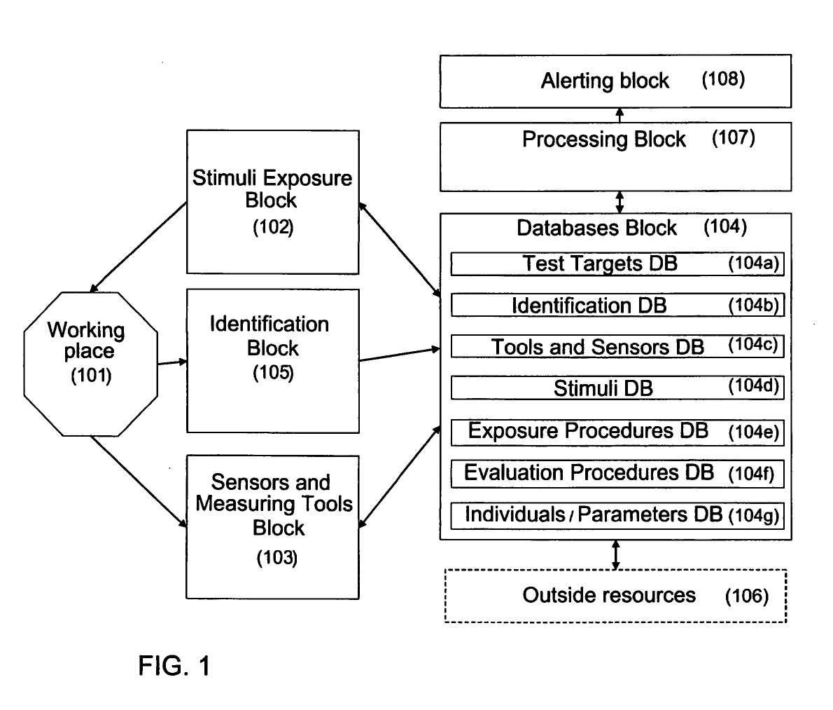

FIG. 1 shows schematically a system for screening and indicating on individuals with malicious and other hidden intent in accordance with an embodiment of the invention.

Working place (e.g. workstation) 101 is connected with Stimuli Exposure Block 102 and Sensors and Measuring Tools Block 103. Working place is equipped with means necessary for fulfillment of a special or routine assignment. Stimuli Exposure Block 102 may comprise different tools providing stimuli exposure to the working place, e.g. sound player, display, biometric input device, etc. Sensors and Measuring Tools Block 103 may comprise variable sensors (e.g. image sensors and recorders, biometric and behavior sensors, sound recorders, etc.) for receiving and registration of individual's psycho-physiological characteristics, including reaction to said stimuli exposure. These sensors may require direct contact with the individual at the working place or operate from a distance without touch (e.g. photographing pupil size, voice analyzing, registration of associative sequence, etc). Stimuli Exposure Block 102 and Sensors and Measuring Tools Block 103 are the functional blocks and may contain dual-purpose tools, which generally consist as part of a working place.

Stimuli Exposure Block 102 and Sensors and Measuring Tools Block 103 are connected with Databases Block 104, comprising several active databases.

Test Targets Database 104a includes potential regions of interest (e.g. terrorists, drug dealers, deceptive employees, etc.), to be selected during the system setup before a test. Identification Database 104b includes data on alleged individuals received from different Outside Resources 106 (e.g. government databases, airway companies' databases, luggage screening systems, credit companies' databases, etc.) as well as personal data on tested individuals. The system may identify the individual with the help of Identification Block 105 or receive the identification data from Outside Resources 106. Personal data on an individual (e.g. origin, religion, profession, traveling information, credit history, etc.) may be considered during the system setup. Identification block may include equipment for documents inspection and/or biometric identification (e.g. Iris Access™ 3000 by LG for pupil photography or DaonEngine for fingerprint control).

Reverting now to block 104, Tools and Sensors Database 104c comprises data about stimuli exposure tools, sensors and measuring tools as well as data concerning current availability of said tools.

Stimuli Database 104d comprises a wide variety of stimuli and data on current availability of said stimuli. The stimuli might be open or concealed, stressing or relaxing, creating conscious or sub-conscious reaction, targeted or general, relevant or irrelevant to the subject of the tests. According to certain embodiment said stimuli do not require an active direct reaction of the tested individual (e.g. there is no need in an active action as to answer a question or to select specific picture as a reaction to stimuli), but do prompt a measurable psycho-physiological (incl. but not limited to biometric and behavioral) reaction.

Exposure Procedures database 104e contains exposure algorithms and scenarios defining type, combination and sequence of stimuli, media and tools for exposure as well as psycho-physiological parameters to be measured. According to the described certain embodiment said stimuli may be any one or any combination of stimuli from Stimuli Database 104d provided with or without human intervention via physical and/or virtual media. Type and time of the stimuli exposure may be random (pseudo random) or scheduled. Exposure Procedures Database may also comprise predefined scenarios of stimuli exposure. Some examples of stimuli combinations:
1) Focused, subconscious, concealed, stressing combination: flashing inside a screen observed by the tested individual a symbol which is relevant only to the threat group and is subconsciously perceived by the individual.
2) General, conscious, open, stressing combination:
   Security inspector posted at the entrance to the working place.

In one of embodiments the stimuli and registered parameters may be selected automatically while neither individual nor human operator may have influence or knowledge about the stimuli and the parameters to be registered. This lack of knowledge of the exact manipulation will counteract a preparatory training which the individual may have trained for to control the reaction when being subjected to a given stimuli.

Evaluation Procedures Database 104f comprises test criteria and predefined discrepancies for different exposure scenarios as well as rules and algorithms for evaluation of discrepancy between registered parameters and test criteria. Said test criteria also include test patterns and rules for their adaptation during the test.

All parameters registered by Sensors and Measuring Tools block 103 are transferred to Individuals/Parameters Database 104g. This database stores data on tested individuals including registered parameters, derivatives thereof, adapted test patterns and results of discrepancy evaluation(s).

The Databases Block 104 may support large number of data variations as well as adjustment for fast changes. The databases adjustment process may be based on data accumulation, analyzing and updating as well as absorption of information from Outside Resources 106. In one of the embodiments, the updating process is subject to human decision and is not fully automated in order to avoid uncontrolled shifting of decision parameters.

Note that the invention is not bound by the databases configuration of FIG. 1, the said data may be organized in consolidated database(s) or divided in another manner.

The Processing Block 107 provides all the necessary processing and management of data stored in the Database Block 104. During the system test setup, the Processing Block 107 defines the Test Framework including selections of stimuli and tools for exposure, selection of sensors and measuring tools, selection of exposure and evaluation procedures. This Test Framework is derived from data stored in the Database Block 104. During the test procedure the Processing Block 107 executes all calculations and data management specified in said Test Framework. If discrepancy matching to a predefined malicious range is discovered, the Processing Block 107 will send notice to the Alerting Block 108, providing, e.g. audio, visual or telecommunication (e.g. SMS or e-mail) indication.

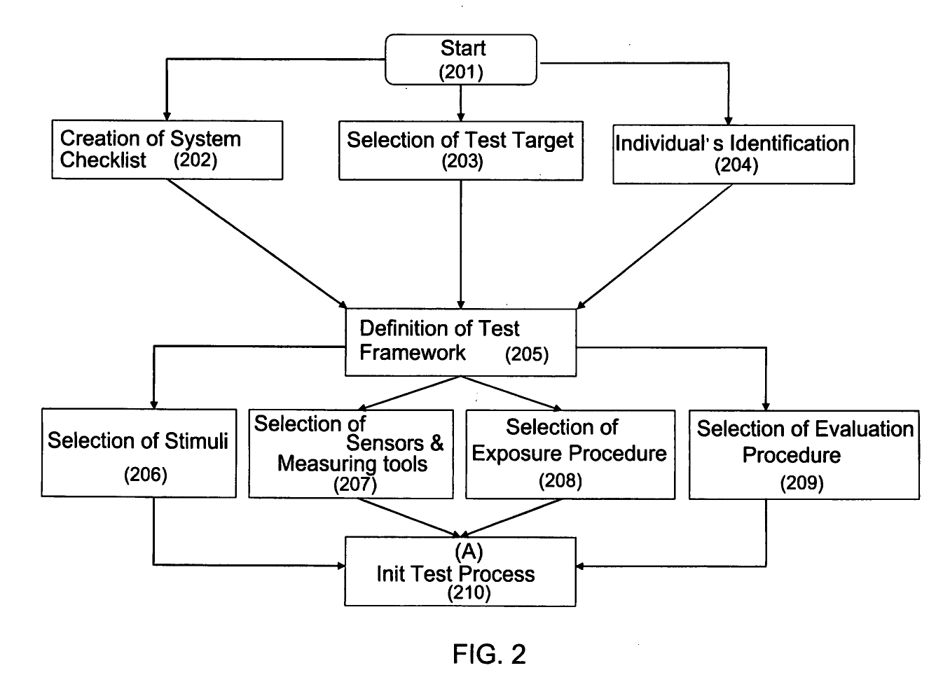

FIG. 2 shows the principal steps of setup operation of the screening process.
With the beginning of the process 201, the Processing Block 105 in FIG. 1 processes the data of currently available sensors, tools, stimuli and test procedures and creates a System Checklist 202. The checklist varies according to the specific requirements and availability of measurement tools and stimuli at each site. The system also provides identification of individual 203 or receives the identification data from outside resources. In parallel, an operator may select the test target(s) 204, e.g. locating of potential drug dealers or terrorists or deceptive employees, etc. Based on these data, said Processing Block defines the Test Framework 205. The defined Test Framework determines which stimuli and exposure tools 206, sensors & measuring tools 207, exposure procedures 208 and evaluation procedures 209 are to be used during the test. For example, if the target is drug dealers or the identified individual is known in the Identification Database 104b in FIG. 1 as an alleged drug dealer, the Test Framework may include drug sniffers, pupil size meter, heroin sensor within the measuring tools 207 and, at least, some of stimuli 206 may be targeted to the drugs-related issues.

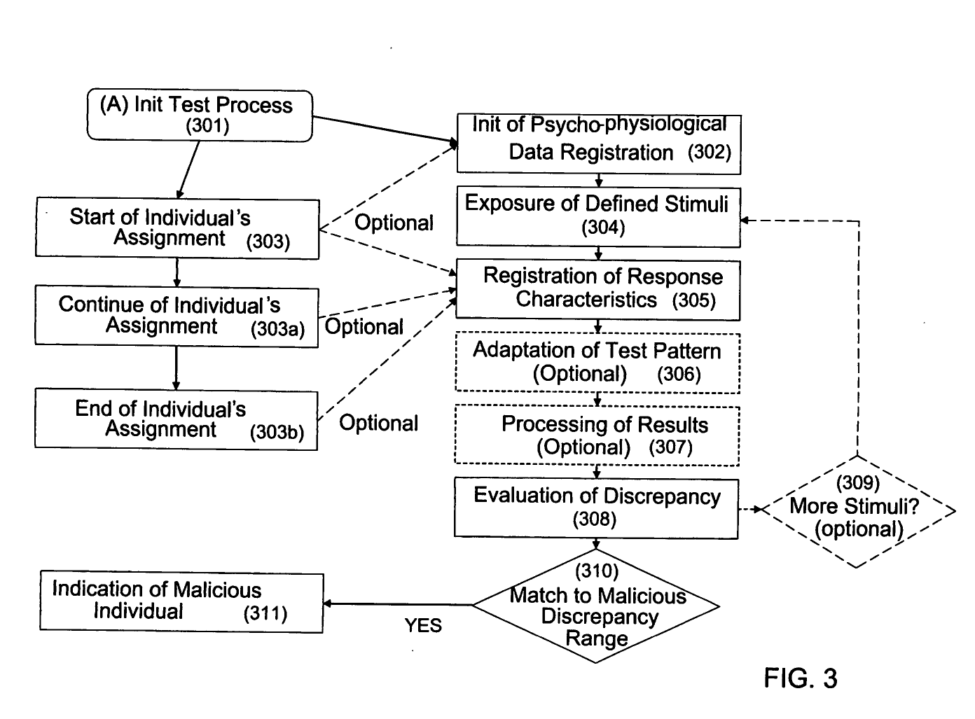

FIG. 3 shows a flow diagram illustrating the principal steps of operating the test in accordance with an embodiment of the invention.

The procedure continues from stage 210 in FIG. 2. The start of the test process 301 and registration of psycho-physiological characteristics 302 are followed by two contemporary flows (“dual task”)—1) individual's fulfillment of special or routine assignment(s) 303-303b and 2) individual's exposure to the stimuli 304 defined in the Test Framework during the setup and in accordance with the procedure defined in said Test Framework.

It should be noted that in other embodiments the individual may start to fulfill the assignment before the start of the test procedure and continue the assignment after the end of the test procedure. Said fulfillment of assignment may include one or several assignments, continuous or with time break(s), identical or of different content and/or nature. For example, in a case of spot screening embodiment, the assignment may be just to fill-in some standard questionnaire, while in a case of continuous screening it may be everyday routine work executed by the individual.

Said exposure of stimuli may be executed during one or several time frames. Said stimuli may be any one or any combination of open or concealed, stressing or relaxing, creating conscious or sub-conscious reaction, relevant and irrelevant to the subject of the test, targeted or general stimuli selected and provided with or without human intervention, provided via physical and/or virtual media. The stimuli do not require an active direct reaction from the tested individual (e.g. there is no need to direct action as to answer a question or select specific picture as a reaction to stimuli) but do prompt a measurable psycho-physiological reaction.

Stimuli within said exposure timeframe(s) may be grouped in dedicated sets, e.g. neutral, control, manipulating, authentication, relaxing set, etc. Stimuli within a timeframe may be of different types and/or different sets. Also some of said timeframes may be targeted and comprise a dedicated set(s) of stimuli. Dedicated set of stimuli may include any of above types of stimuli exposing with a specific purpose. For example, control set of stimuli is dedicated to cause a constant (or almost constant with predefined deviation) reaction of individual which later on can be used as a reference of his normal state reaction (e.g. to indicate any countermeasures dedicated to reduce the responsiveness), neutral set of stimuli is a subset of control stimuli and are supposed not to cause a reaction in a normal state of tested individual, authentication set of stimuli is dedicated to cause a predefined reaction specific to tested individual and enabling his/her authentication, etc.

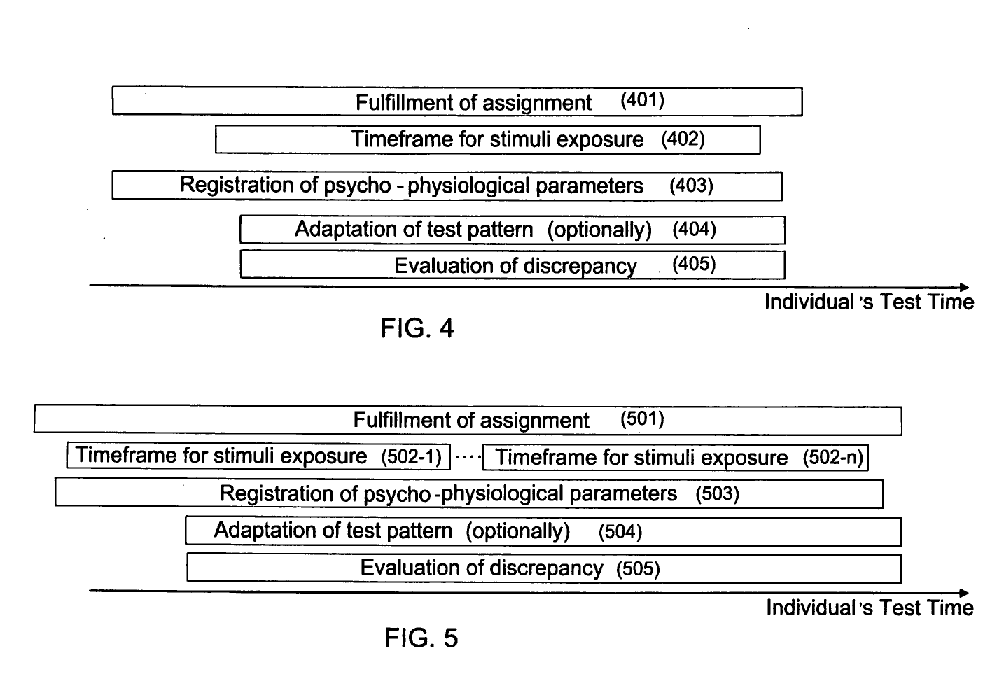

The appearance and duration of exposure time frame(s) as well as type and time of appearance of the stimuli within said exposure time frame(s) may be random (pseudo random) or scheduled. FIGS. 4-6 illustrate in a non-limiting manner some specified embodiments of time sequence of test operation. Some of the test scenarios will be illustrated in examples below.

The system will continue to register psycho-physiological parameters including the parameters of response to said exposure 305. Optionally, the system may also register the parameters of fulfillment of assignment, e.g. speed of typing, pauses, typing mistakes, etc.

The further step of test pattern adaptation 306 is optional and defined in said Test Framework. Generally, the test pattern may be defined in advance based on prior knowledge on an individual or a threat group. The adaptive pattern comprises predefined types of parameters whereas their values (or part of them) are adapted to the tested individual according to the registered parameters. Usually the pattern adaptation is implemented to enable evaluation of discrepancy between parameters registered under different conditions, e.g. discrepancy between response to neutral and manipulating stimuli, control and manipulating stimuli, etc. For example, adaptive pattern may comprise a set of predefined psycho-physiological parameters (e.g. blood pressure and pupil size) to be measured during control exposure; values of these parameters are measured during said exposure and put in the pattern during the adaptation process.

The further step of processing results 307 is optional, defined in said Test Framework and comprises processing of the registered parameters before comparing the derivatives thereof with test criterion. Such processing, for example, may include statistical analysis, correlation analysis, etc. The processing may include, for example, statistical analysis of overall registered parameters or correlation analysis of parameters registered during different exposure timeframes or parameters registered during different dedicated sets of stimuli, etc.

Evaluation of discrepancy 308 is the step of comparing the registered parameters or derivatives thereof with a test criterion. Test criterion is defined in the Test Framework. Test criterion may be Boolean or quantified, may refer to a specific registered parameter, group of parameters or derivative thereof. It can also be a test pattern including several parameters (and/or derivative thereof) and relations between them. The test pattern may be predefined or adaptive as described above.

According to the evaluation results, the system may optionally, e.g. if there is not enough data for reliable evaluation, consider whether to repeat the test process 309.

During the next step 310 the evaluated discrepancy (if any) between registered parameters and the test criterion is compared with the malicious discrepancy range, which is defined in the Test Framework. If the discrepancy matches said malicious discrepancy range, the system will provide indication of hidden intent. Said indication may originate any type of alert, depending on a specific embodiment of the invention.
Those versed in the art will readily appreciate that the invention is not bound by this specific sequence of operations of this non limiting example.

FIGS. 4-6 illustrate in non-limiting manner flow charts of generalized time sequence of test operating, in accordance with some embodiments of the invention.

In the embodiment illustrated by FIG. 4 the fulfillment of the assignment 401 and registration of psycho-physiological parameters 403 start simultaneously, while exposure time frame 402 may start simultaneously or later. Adaptation of test pattern 404 is optional and discrepancy evaluation 405 as well may start any time during the test in accordance with the Test Framework.

The delay between the start of assignment and the start of exposure time frame enables, when necessary, to register parameters for said optional adaptation of the test pattern before exposure of any stimulus.

The embodiments illustrated by FIG. 4 may be used, as non-limiting example, for spot-screening purposes. These purposes can be illustrated, for example, to locate drug smugglers in an airport. For clarity, the description of the process will occasionally refer also to operational steps depicted in FIGS. 2 and 3.
1) After individual enters the airport he/she will pass identification procedure (204 in FIG. 2) which may include documents inspection and biometric tests (for example: pupil photography system—IrisAccess™ 3000 by LG; fingerprints and access control systems—DaonEngine and DaonAccess), the transparent and sensing procedures (e.g. X-Ray and Raman Spectroscopy analysis), as well as intelligence clearance. Given the results, the system starts the test procedure in accordance with specific embodiment of the current invention.
2) Test system database will define the Test Framework (205 in FIG. 2) in accordance with the target, e.g. drug smugglers (203 in FIG. 2), identification and the system checklist (202 in FIG. 2). Said Test Framework definition may also take into account individual's personal data (204 in FIG. 2) (e.g. origin, religion, profession, traveling information, etc.).
3) The individual will be asked to fill in some computerized questionnaire (401 in FIG. 4). Simultaneously the system starts to register (403 in FIG. 4) the biometric parameters (e.g. temperature and heart rate via biometric mouse, pupil size and facial expressions through image recorders inside the screen, etc.). Later this registered parameter may be used for adaptation of the test pattern (404 in FIG. 4) which may include, for example, all said parameters registered before exposure of the first stimulus.
4) Contemporary to the questionnaire (fulfillment of assignment) the system will start the stimuli exposure time frame (402 in FIG. 4). The individual, as well as the operators of the system, do not know whether, when or which stimuli will appear. Thus, the individual is not able to plan his reaction in order “to please” the system and modify the results. For example, the following stimuli may appear during the exposure time frame: a) “Currently airport security has identified two drug smugglers” announced via sound player, b) later the system flashes a short frame (concealed, sub conscious stimulus) showing the picture of a drug baron who is suspected to be connected with the smugglers c) some time after the name of a drug baron will appear on the screen, etc.
5) The system will compare the parameters registered during the exposure of stimuli with said adapted pattern and evaluate discrepancy (405 in FIG. 4). The registered data may be processed (307 in FIG. 3) with the help of statistical analysis based on Within Subject Changes analyzing tools, which enable revision of minor changes of parameters in minimum test repetitions. If the changes in the registered parameters (e.g. rise or decline in heart pace, changes in pupil size, facial expressions and temperature) exceed some predefined value (310 in FIG. 3), the individual may be indicated as suspicious (311 in FIG. 3) and will be sent for a more detailed inspection. More specifically, the system may alert to a suspect in the case that the individual was graded (310 in FIG. 3) with a mark that is defined as suspicious (for example=>0.8) by one or more of the test tools, while the grade for evaluation is calculated according to the formula:
   Z=sum of (K1 to Kn)/n, when:
   Z—Evaluation grade
   K—Ascription grade in specific test
   N—Number of tests
In case of a lower grade in one or more of the tests, the criterion for decision is a sum of more than 0.7 (also by way of example). In case of lack of response in one of the tests (for example, a deaf person in a hearing test), the system decides according to M parameters out of N tests that were activated. In order to increase reliability of the test, the results may be analyzed in a Fuzzy Logic process. In this case the results are not absolute, but give a general direction as to whether or not the subject belongs to a threat group.
The results may be analyzed in at least one of three levels: against general population data, within the subject, that allows detecting the changes resulting from the stimuli exposure, and against prior data gathered on the tested individual by similar systems as in the past. The stimuli are intended to target individuals who understand the meaning of the stimuli and can react to it, but the stimuli do not require any active direct reaction. (For example, stressing manipulations: a picture of a drugs syringe and a policeman—targeting a drugs dealer, a picture of an Islamic leader or symbol—targeting Islamic terrorists; for a calming manipulation: a picture of a peaceful escape resort in the islands—targeting a suspect on the run).
Those versed in the art will readily appreciate that the invention is not bound by this specific sequence of operations of this non limiting example.

In the embodiment illustrated by FIG. 5 assignment fulfillment 501, by way of example, starts prior to registration of psycho-physiological parameters 503. The main difference in this embodiment is that the stimuli are exposed during several time frames (502-1 to 502-n). Said time frames can be of different duration with different intervals between them. Such embodiment enables easy implementation of repetitive test procedures. The intervals between exposure timeframes increase the stickiness of the individual in fulfilling the assignment(s) as well as increase the versatility for optional adaptation of the test pattern 504. Said optional adaptation as well as discrepancy evaluation 505 may start any time during the test, in accordance with the Test Framework.

The embodiments illustrated by FIG. 5 may be used, as non-limiting examples, for progressive-screening purposes.

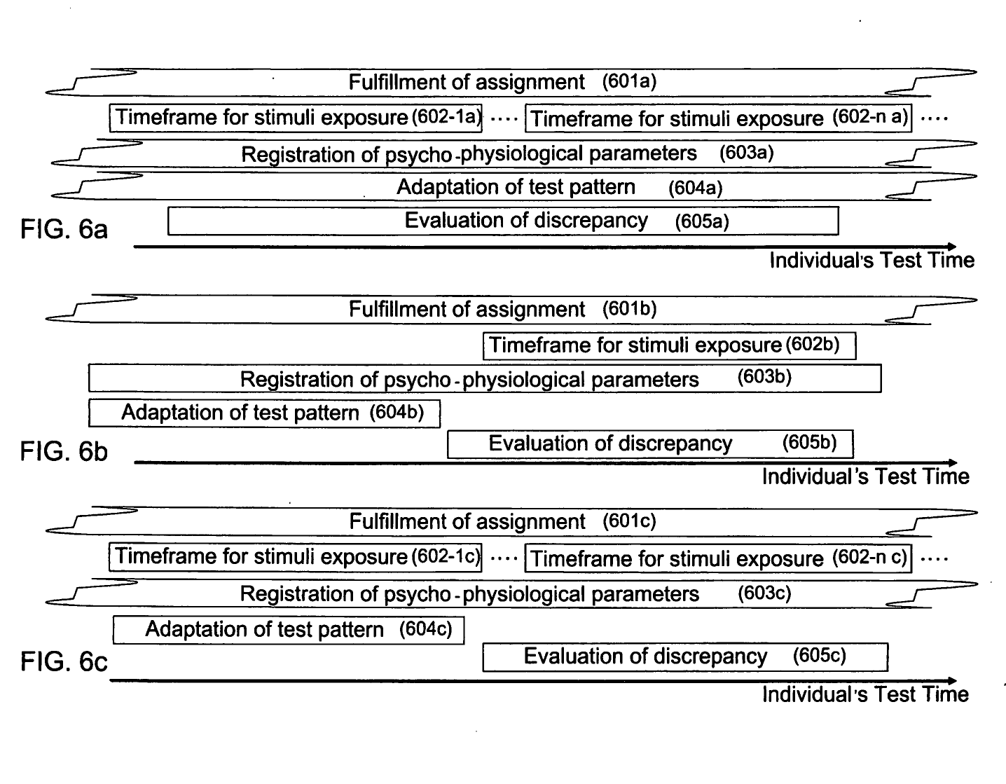

FIGS. 6a-6c illustrate some variations of embodiment when the individual starts to fulfill the assignment (601a, 601b, 601c) before the start of the test.

In the variations illustrated in FIG. 6a and FIG. 6c registration of psycho-physiological parameters (603a and 603c) also start before commencing the test while in the variation of FIG. 6b the registration 603b starts subsequent to commencement of the test. The stimuli are exposed within one timeframe 602b in the variation on FIG. 6b and within several timeframes (602-1a to 602-na, 602-1c to 602-nc) in the variations illustrated by FIG. 6a and FIG. 6c. The adaptation of test pattern starts before the test and continues after the test (604a as in FIG. 6a), starts after the test and is completed before the first exposure timeframe (604b as in FIG. 6b) and provides contemporary test with one exposure timeframe (604c as in FIG. 6c). Evaluation of discrepancy (605a, 605b, 605c) may start any time during the test in accordance with the Test Framework. Adaptation of test pattern as well as evaluation of discrepancy may be repeated progressively.

The embodiments illustrated by FIGS. 6a-6c may be regarded as non-limiting examples, for continuous-screening purposes. These purposes can be illustrated, for example, by trustworthiness tests for employees of sensitive organizations (e.g. banks, governmental institutes, etc.).
1) When starting work, an employee, e.g. a bank clerk responsible for loans' confirmation, may be tested by filling a biographic and associated questionnaire (for example: Rapaport, D., Gill, M, and Schafer, R. (1946). Diagnostic Psychological Testing: A Battery of Tests—b The Theory, Statistical Evaluation and Diagnostic Application, Chicago: Year Book Pub) which may include biometric and behavior measuring tools. Similar tests can be repeated periodically or randomly. The registered data may be used to create control sets of stimuli with test pattern adapted for said employee (306 in FIG. 3)
2) Employee's working place (101 in FIG. 1) may be equipped with several tools for stimuli exposure (102 in FIG. 1) and several sensors allowing registration of psycho-physiological parameters of employee (103 in FIG. 1), e.g. with the camera inside the computer screen for registration of pupil size and IR eyes photography identifying changes in blood flow within the peripheral blood vessels around the eye, facial expressions and temperature; respiration and movement sensor inside a chair cushion, biometric mouse, etc. Working place equipment also may be used for test purposes (e.g. computer screen can be used for stimuli exposure and communication microphone can be used as a sensor recording the employees' voice).
3) The employee will be continuously tested, e.g. for fraud, contemporary with the fulfillment of his routine assignment. The employee may or may not be aware that he is undergoing a test. The test may include a special set of stimuli dedicated for authentication of an individual currently working at the working place as a tested employee (e.g. 602-c in FIG. 6c). This authentication includes evaluation of discrepancy (e.g. 605-c in FIG. 6c) between test pattern previously adapted for the employee (e.g. 604-c in FIG. 6c) and the parameters registered during exposure of said dedicated set of stimuli. In a similar manner the special set of stimuli may be used to control countermeasure attempts by employee. If employee supposes that he is undergoing a test and uses some medical treatment to reduce the response, a discrepancy between his current response to exposure of control set of stimuli and test pattern adapted according to his previous response to the same set will indicate a potential hidden intent.
4) Test Framework will define the appearance and duration of exposure timeframes and what stimuli and when to be exposed within said timeframes. The employee, as well as examiner, does not know whether, when or which stimuli will appear. Thus, the individual is not able to plan his reaction in order “to please” the system and modify the results.
5) The system will compare the parameters registered during the exposure of stimuli with said pattern adapted to the employee and evaluate discrepancy. If the changes in the registered parameters exceed some predefined value, the individual may be indicated as suspicious and be sent for more detailed inspection.
6) The procedure can be repeated several times with the same or different sets of stimuli so as statistically determine whether there is indication of hidden intent.
   Those versed in the art will readily appreciate that the invention is not bound by this specific sequence of operations of this non limiting example.

One of embodiments of the present invention was tested in a following experiment.

48 tested individuals were organized in 12 groups for competition in solving computerized logic quizzes.

Each group comprised one “cheater”, one “collaborator” and two “naive” participants:
“Cheater”—an individual, who experienced distress while trying to solve a very difficult quiz, and finally had to use illegal clues in order to accomplish his mission.
“Collaborator”—subject, who was secretly recruited to collaborate with the experimenters
“Naïve” subjects

All individuals were asked to solve computerized logic quizzes as part of their group task with no communication within the group and between the groups. The quizzes were presented to the individuals over a personal computer screen, and without their knowledge, each one of them faced a different difficulty level:
“Collaborator”—faced an easy quiz, that could be solved in a short time, thus became a Stress Inducer for the other team members (other subjects)
“Cheater”—faced a very difficult quiz that a reasonable person would not be able to solve within the test given timeframe, unless he uses illegal clues.
“Naïve”—faced a reasonable quiz, that could be solved within the test timeframe—some time after the easy quiz (collaborator's) and before the difficult one (cheater's)

After completing of said task the participants were asked to answer a feedback questionnaire over the computer screen (assignment 303 in FIG. 3). While occupied with this assignment all individuals were equally exposed to targeted stimuli (304 in FIG. 3) which were designed to create measurable Differential Response, only within Targeted Subject (“Cheater” or “Collaborator”):
“Cheater” stimulus—an announcement heard by all group members, saying “we believe that one of the team members has been cheating”
“Collaborator” stimulus—a discussion overheard by all members of the group, revealing the fact that one of them is a collaborator that is making more money and getting easier quizzes.

The following biometric parameters were registered (305 in FIG. 3) during the experiment and evaluated for identification (308 in FIG. 3) as “Cheater”, “Collaborator” or “Naïve” individuals:
PPG (Pulse Pressure) and HR (Heart Rate)—by Photo-Platismograph, and DAS3 system, manufactured by Atlas Research.
Respiration and movements—by DAS3 system, manufactured by Atlas Research.
GSR (Galvanic Skin Response) and ASR (GSR derivative)—by DAS3 system, manufactured by Atlas Research
Voice analysis—recorded, and than analyzed by parameters measured by LVA6.5 system, manufactured by Nemsysco.
Temperature changes around eyes—by FLIR camera and software, EYE R321, manufactured by OPGAL.

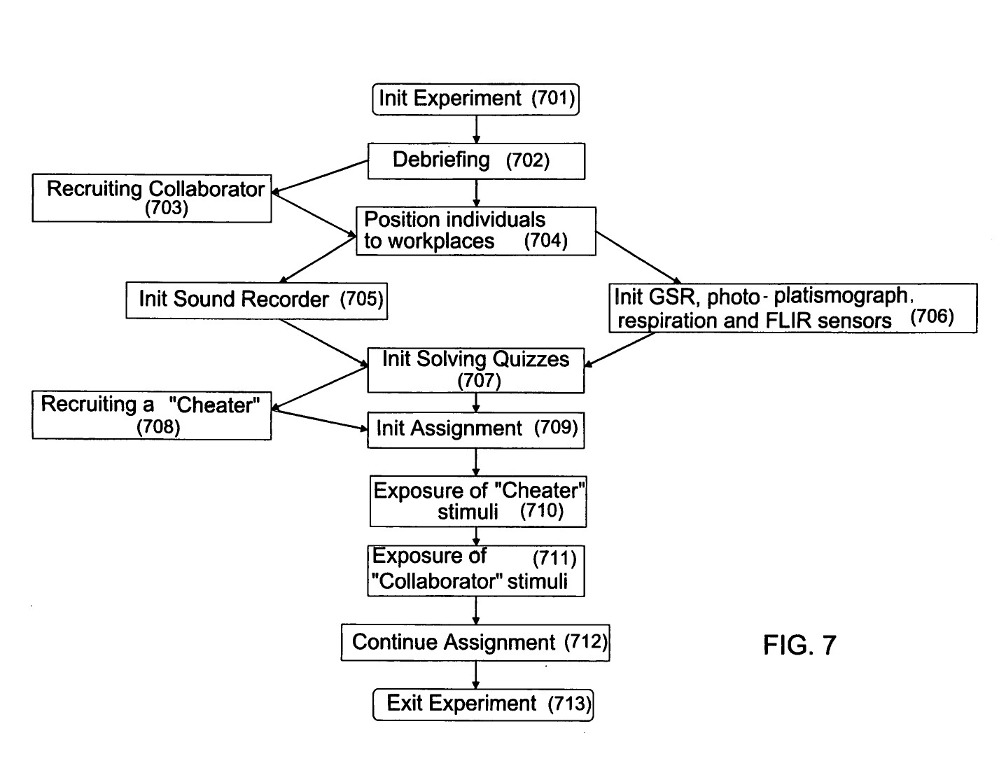

FIG. 7 illustrates a flow diagram of the principal steps of said experiment.

For clarity, the description of FIG. 7 will occasionally refer also to operational stages depicted in FIGS. 2 and 3.

After initiation 701 (301 in FIG. 3) of the experiment each group underwent debriefing 702. Then each participant attended a personal interview, which was used by the experimenter as an opportunity for recruiting 703 one of them as a “Collaborator”, without his friends' awareness. The “Collaborator”, in return for agreeing to collaborate, was promised a higher reward and easier quizzes. The individuals entered the test room, where they were placed in personal test stations 704 and connected to the measuring system 705 and 706 (pattern adaptation 306 in FIG. 3). After that the participants started to solve the quizzes 707 while the progress of each of them was indicated by four counters over their screens. Due to the different difficulty levels, the “Collaborator” succeeded to solve it first, and his counter stopped; the two “Naives” finished second and third—and their counters stopped as well; only the future “cheater” was “stuck”—counter kept running—not being able to solve. Then, through his personal earphones, he overheard an “accidental discussion” between the experimenters that gave the correct solution. The individual used the clues, of course, succeeded to solve his quiz and stop the counter and thus was designated a “Cheater” 708.

Once the group successfully solved the quizzes the participants were asked to fulfill an assignment 709 (303 in FIG. 3)—to answer a feedback questionnaire over the computer screen.

Contemporary with that assignment all individuals were exposed to stimuli 710 (first dedicated set of stimuli, 304 in FIG. 3) targeted to identify the “Cheater” while their biometric characteristics were recorded and synchronized over timeline (305 in FIG. 3). (An announcement: “seems that one of you “cheated” a little, and used clues he accidentally overheard”). A short time after, all individuals were exposed to stimuli 711 (second dedicated set of stimuli, 304 in FIG. 3) targeted to identify the “Collaborator”, while their biometric characteristics were recorded and synchronized over timeline (305 in FIG. 3). (An “accidental” discussion between experimenters: “did you see that collaborator? He is not that smart—not only he got more money, his quizzes were much easier!”)

The individuals continued to answer the questioner 712 (303a-b in FIG. 3) until the end 713 of the test.

The analysis of test results (308 in FIG. 3) was done by a “blind” analyzer that received the subjects' biometric recordings, synchronized over timeline with “Cheater's” and “Collaborator's” targeted stimulus. All 48 data files were presented to the analyzer in a complete random manner (1 out of 48), without any background information about the individuals—as to which group they belonged, their gender, position, etc. The analyzer evaluated each individual's registered parameters separately and indicated whether the individual is a “Cheater”, a “Collaborator”, or a “Naïve” (311 in FIG. 3).

Hit rate was 46 out of 48 (96%, n+=46, n−=2, p<0.0001). False negative rate was 1 out of 48 (2%), false positive rate was 0 out of 48 (0%) and 1 subject response was not clear, thus required further inspection.

It is to be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

It is also to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present invention.

Those skilled in the art will readily appreciate that various modifications and changes can be applied to the embodiments of the invention as hereinbefore described without departing from its scope, defined in and by the appended claims.

1-50. (canceled)

51. A method for computerized indicating individuals with hidden intent, the method comprising:
(a) exposing an individual to one or more stimuli within at least one exposure timeframe while said timeframe is contemporary with individual's fulfillment of at least one stated assignment and said stimulus does not require an active direct reaction from the individual and is intended to cause a psycho-physiological reaction;
(b) registration of individual's psycho-physiological characteristics giving rise to registered parameters;
(c) comparing the registered parameters or derivatives thereof with a test criterion;
(d) providing indication of hidden intent in case of predefined discrepancy.

52. The method of claim 51 wherein said hidden intent is malicious.

53. The method of claim 51 wherein at least one of said stimuli does not form a part of said assignment.

54. The method of claim 51 wherein said psycho-physiological characteristics include a behavioral reaction.

55. The method of claim 51 wherein at least one of said stimuli is a member of a group comprising stimuli of the types as open stimuli, concealed stimuli, stressing stimuli, relaxing stimuli, stimuli creating conscious reaction, stimuli creating sub-conscious reaction, targeted stimuli, general stimuli.

56. The method of claim 51 wherein the provision of at least one of said stimuli is fully automated.

57. The method of claim 51 wherein at least one of said stimuli is a member of a group comprising stimuli provided via physical and virtual media.

58. The method of claim 51 wherein at least part of said stimuli are grouped within the exposure timeframe in at least one dedicated set.

59. The method of claim 58 wherein at least one set of stimuli is dedicated to individual's authentication.

60. The method according to claim 58 wherein at least one set of stimuli is dedicated to indication of countermeasures by the individual.

61. The method of claim 51 comprising random or pseudo-random selection of at least one type of said stimuli.

62. The method of claim 51 comprising random or pseudo-random exposure of at least one of said stimuli within the exposure timeframe.

63. The method of claim 51 comprising scheduled exposure of at least one of said stimuli within the exposure timeframe.

64. The method of claim 51 wherein said test criterion includes a test pattern; the method further comprising adapting said test pattern to the individual according to registered parameters.

65. The method of claim 64 comprising adapting said test pattern to parameters registered before the first exposure time frame.

66. The method of claim 64 comprising adapting said test pattern to parameters registered between exposure time frames.

67. The method of claim 64 comprising adapting said test pattern to parameters registered during exposure of a dedicated set of stimuli.

68. The method of claim 51 wherein appearance and continuity of at least one exposure timeframe is random or pseudo-random.

69. The method of claim 51 wherein appearance and continuity of at least one exposure timeframe is scheduled.

70. The method of claim 51 wherein said assignments include answering questions and at least one stimulus comprising flashing at least one picture not related to the content of the questions.

71. A method for computerized indicating individuals with hidden intent, the method comprising:
(a) exposing an individual to at least two sets of stimuli within one or more exposure timeframe, while first set of stimuli is dedicated to cause a known in advance individual's response and the second set is dedicated to indicate a hidden intent;
(b) registration of psycho-physiological characteristics giving rise to registered parameters;
(c) comparing the parameters or derivatives thereof registered during the exposure of the first set with the known in advance individual's response;
(d) comparing the parameters or derivatives thereof registered during the exposure of the second set with a test criterion;
(e) providing an indication of hidden intent in case of predefined discrepancy.

72. The method of claim 71 wherein said comparison with the known in advance individual's response is provided for the purpose of an individual's authentication.

73. The method of claim 71 wherein said comparison with the known in advance individual's response is provided for the purpose of indicating countermeasures by the individual.

74. The method of claim 71 wherein each of said exposure timeframes is contemporary with individual's fulfillment of at least one stated assignment.

75. The method of claim 71 wherein said first and second sets of stimuli are exposed in different exposure timeframes.

76. The method of claim 71 wherein said first and second sets of stimuli are exposed within the same exposure timeframes.

77. The method of claim 71 wherein said hidden intent is malicious.

78. The method of claim 71 wherein said psycho-physiological characteristics include a behavioral reaction.

79. The method of claim 71 wherein at least one of said stimulus is a member of a group comprising stimuli of the types as open stimuli, concealed stimuli, stressing stimuli, relaxing stimuli, stimuli creating conscious reaction, stimuli creating sub-conscious reaction, targeted stimuli, general stimuli.

80. The method of claim 71 wherein provision of at least one of said stimuli is fully automated.

81. The method of claim 71 wherein at least one of said stimulus is a member of a group comprising stimuli provided via physical and virtual media.

82. The method of claim 71 comprising random or pseudo-random selection of a type of at least one of said stimuli.

83. The method of claim 71 comprising random or pseudo-random exposure of at least one said set of stimuli within the exposure timeframe.

84. The method of claim 71 comprising scheduled exposure of at least one said set of stimuli within the exposure timeframe.

85. The method of claim 71 wherein said test criterion includes a test pattern; the method further comprising adapting said test pattern to the individual according to registered parameters.

86. The method of claim 71 wherein said known in advance individual's response includes a test pattern; the method further comprising adapting said test pattern to the individual according to parameters registered in advance.

87. The method of claim 71 wherein appearance and continuity of at least one exposure timeframe is random or pseudo-random.

88. The method of claim 71 wherein appearance and continuity of at least one exposure timeframe is scheduled.

89. A system for computerized indicating individuals with hidden intent, the system comprising:
(a) a working place for fulfillment of at least one stated assignment;
(b) a stimuli exposure block configured to exposure at least one stimuli to said working place within at least one exposure timeframe while said timeframe is contemporary with individual's fulfillment of at least one stated assignment and said stimulus does not require an active direct reaction from the individual and is intended to cause a psycho-physiological reaction;
(c) sensors and measuring tools block configured to register individual's psycho-physiological characteristics giving rise to registered parameters;
(d) a processor configured to compare the registered parameters or derivatives thereof with a test criterion and to facilitate indicating a hidden intent in case of predefined discrepancy.

90. A computer program product comprising a computer useable medium having computer readable program code embodied therein for indicating individuals with hidden intent, the computer program product comprising:
(a) computer readable program code for causing the computer to expose an individual to at least one stimulus within at least one exposure timeframe while said timeframe is contemporary with individual's fulfillment of at least one stated assignment and said stimulus does not require an active direct reaction of the individual and is intended to cause a psycho-physiological reaction;
(b) computer readable program code for causing the computer to register individual's psycho-physiological characteristics giving rise to registered parameters;
(c) computer readable program code for causing the computer to compare the registered parameters or derivatives thereof with a test criterion;
(d) computer readable program code for causing the computer to provide indication of hidden intent in case of predefined discrepancy.

91. A computer program product comprising a computer useable medium having computer readable program code embodied therein for indicating individuals with hidden intent the computer program product comprising:
(a) computer readable program code for causing the computer to expose an individual to at least two sets of stimuli within one or more exposure timeframe, while first set of stimuli is dedicated to cause a known in advance individual's response and the second set is dedicated to indicate a hidden intent;
(b) computer readable program code for causing the computer to register psycho-physiological characteristics giving rise to registered parameters;
(c) computer readable program code for causing the computer to compare the parameters or derivatives thereof registered during the exposure of the first set with the known in advance individual's response;
(d) computer readable program code for causing the computer to compare the parameters or derivatives thereof registered during the exposure of the second set with a test criterion;
(e) computer readable program code for causing the computer to provide an indication of hidden intent in case of predefined discrepancy.
